(19) United States
(12) Patent Application Publication
(10) Pub. No.: US 2019/0307347 A1
(43) Pub. Date: Oct. 10, 2019

(54) SYSTEMS AND METHODS FOR PROCESSING BIOLOGICAL DATA SIGNALS USING AN IMPLANTABLE MEDICAL DEVICE

(71) Applicant: PACESETTER, INC., Sytlmar, CA (US)

(72) Inventor: Dean ANDERSEN, San Jose, CA (US)

(21) Appl. No.: 15/947,681

(22) Filed: Apr. 6, 2018

(51) Int. Cl. A61B 5/04; A61B 5/042; A61B 5/00

(52) U.S. Cl. CPC A61B 5/04014; A61B 5/7203; A61B 5/042

(57) ABSTRACT

The present disclosure provides systems and methods for processing biological data signals. An implantable medical device includes a sensing component that acquires a raw biological data signal. A filtering component communicatively coupled to the sensing component filters the raw biological data signal to generate a filtered biological data signal by ignoring signal values that fall within an amplitude window defined between an upper threshold and a lower threshold. A processing and storage component communicatively coupled to the filtering component stores the filtered biological data signal.

### FIELD OF THE DISCLOSURE

The present disclosure relates generally to implantable medical devices, and more specifically, to efficiently acquiring and processing biological data signals using an implantable medical device.

### BACKGROUND ART

Implantable medical devices are commonly used to acquire and store biological data signals, such as cardiac signals and neurological signals. For example, the biological data signals may include an intra-cardiac electrocardiogram (IEGM). At least some known implantable Medical devices use a time-based sampling system, in which all biological data is sampled at a fixed time step. This fixed time step generally corresponds to a sample rate of an analog-to-digital converter (ADC) used by the implantable medical device.

The acquired signal is generally processed to extract features of interest (e.g., amplitude information, morphology features, QRS intervals). Further, the acquired signal is also sent to a memory device for storage. If an event of interest is detected, the data stored before and after the event of interest can be retrieved and analyzed.

Notably, for at least some known implantable medical devices, the majority of time spent processing and storing data is of limited clinical relevance, as clinical events of interest happen relatively infrequently. This results in wasted energy and decreased longevity for the implantable medical device, as unneeded data is processed at a high percentage of overall time steps. However, even a relatively small amount of power savings can result in substantially improved longevity. For example, a savings of 500 nano Watts (nW) for an implantable cardiac monitor (ICM) may result in a few months of additional longevity. Accordingly, it would be desirable to provide an implantable medical device that more efficiently processes and stores biological data signals based on the content of the biological data signals.

### BRIEF SUMMARY OF THE DISCLOSURE

In one embodiment, the present disclosure is directed to an implantable medical device. The implantable medical devices includes a sensing component configured to acquire a raw biological data signal, a filtering component communicatively coupled to the sensing component, the filtering component configured to filter the raw biological data signal to generate a filtered biological data signal, wherein to filter the raw biological data signal, the filtering component is configured to ignore signal values that fall within an amplitude window defined between an upper threshold and a lower threshold, and a processing and storage component communicatively coupled to the filtering component, the processing and storage component configured to store the filtered biological data signal.

In another embodiment, the present disclosure is directed to a method for processing biological data signals acquired using an Implantable medical device. The method includes acquiring, using a sensing component of the implantable medical device, a raw biological data signal, filtering, using a filtering component of the implantable medical device, the raw biological data signal to generate a filtered biological data signal, wherein the filtering comprises ignoring signal values that fall within an amplitude window defined between an upper threshold and a lower threshold, and storing the filtered biological data signal using a processing and storage component of the implantable medical device.

The foregoing and other aspects, features, details, utilities and advantages of the present disclosure will be apparent from reading the following description and claims, and from reviewing the accompanying drawings,

### BRIEF DESCRIPTION OF THE DRAWINGS

FIG. 1 is a block diagram of one embodiment of an implantable medical device (IMD).

FIG. 2 is a block diagram of one embodiment of a computing device that may be used with the IMD shown in FIG. 1.

FIG. 3A is a plot of a raw biological data signal acquired by the sensing component of the IMD shown in FIG. 1,

FIG. 3B is a plot of a filtered signal generated by filtering the raw biological data signal using the filtering component of the IMD shown in FIG. 1.

FIG. 4 is a circuit diagram of one embodiment of a filtering architecture that may be used to implement the filtering component of the IMD shown in FIG. 1.

FIG. 5 is a circuit diagram of another embodiment of a filtering architecture that may be used to implement the filtering component of the IMD shown in FIG. 1.

FIG. 6 is a circuit diagram of a threshold determination circuit that may be used to determine a threshold value for the filtering architecture shown in FIG. 5.

FIG. 7A is a plot of a raw biological data signal acquired by the sensing component of the IMD shown in FIG. 1.

FIG. 7B is a plot of a filtered signal generated by filtering the raw biological data signal using the filtering component of the IMD shown in FIG. 1.

Corresponding reference characters indicate corresponding parts throughout the several views of the drawings.

### DETAILED DESCRIPTION OF THE DISCLOSURE

The present disclosure provides systems and methods for processing biological data signals. An implantable medical device includes a sensing component that acquires a raw biological data signal. A filtering component communicatively coupled to the sensing component filters the raw biological data signal to generate a filtered biological data signal by ignoring signal values that fall within an amplitude window defined between an upper threshold and a lower threshold. A processing and storage component communicatively coupled to the filtering component stores the filtered biological data signal.

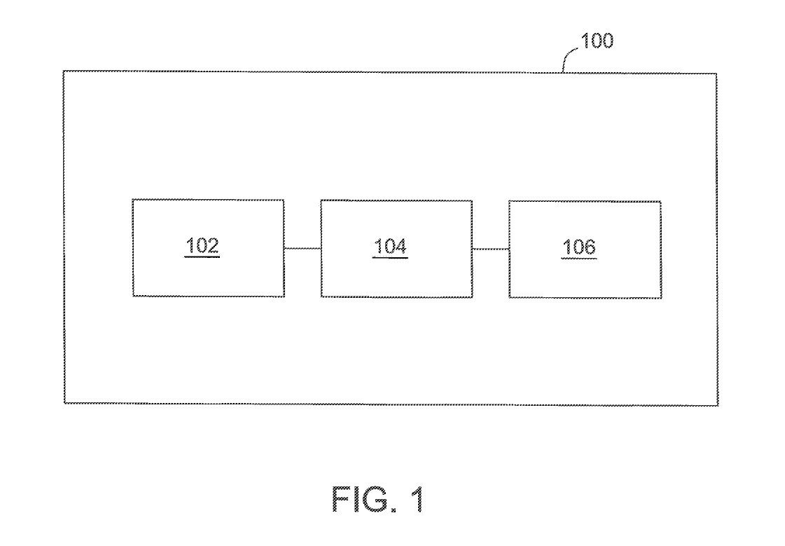

FIG. 1 is a block diagram of one embodiment of an implantable medical device (IMD) 100. IMD 100 is implantable in a patient and operable to acquire:, process, and record biological data signals. For example, in one embodiment, IMD 100 is one or more implantable cardiac monitors (ICM) operable to acquire, process, and record electrocardiograms (ECGs). In another embodiment, IMD 100 may be one or more implantable pacemakers operable to acquire, process, and record intra-cardiac electrocardiograms (IEGMs). Alternatively, IMD 100 may be any suitable implantable medical device capable of capturing any suitable biological data signals. For example, in some embodiments, IMD 100 captures accelerometer signals, neurological signals (e.g., deep brain sensing signals), pressure signals, temperature signals, etc.

As shown in FIG. 1, IMD 100 includes a sensing component 102, a filtering component 104, and a processing and storage component 106. Filtering component 104 is communicatively coupled to sensing component 102 and processing and storage component 106. Sensing component 102 acquires raw (i.e., unfiltered) biological data signals from the patient. Sensing component 102 includes any device capable of capturing or measuring desired biological data signals. For example, sensing component 102 may include one or more sensing electrodes for sensing cardiac and/or neurological signals.

Filtering component 104 filters the raw biological data signals acquired by sensing component, as described in detail herein. Accordingly, filtering component 104 generates filtered biological data signals. Processing and storage component 106 then further processes and/or stores the filtered biological data signals, as described in detail herein.

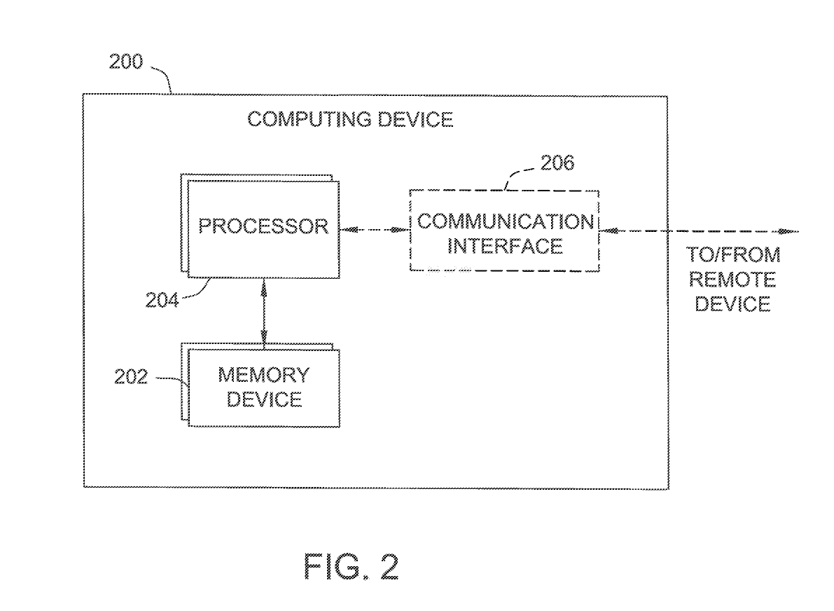

Filtering component 104 and/or processing and storage component 106 may be implemented using a computing device. For example, FIG. 2 illustrates one embodiment of a computing device 200 that may be used with IMD 100.

In this embodiment, computing device 200 includes at least one memory device 202 and a processor 204 that is coupled to memory device 202 for executing instructions. In some embodiments, executable instructions are stored in memory device 202. In the illustrated embodiment, computing device 200 performs one or more operations described herein by programming processor 204. For example, processor 204 may be programmed by encoding an operation as one or more executable instructions and by providing the executable instructions in memory device 202.

Processor 204 may include one or more processing units (e.g., in a multi-core configuration). Further, processor 204 may be implemented using one or more heterogeneous processor systems in which a main processor is present with secondary processors on a single chip. In another illustrative example, processor 204 may be a symmetric multi-processor system containing multiple processors of the same type. Further, processor 204 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 202 is one or more devices that enable information such as executable instructions and/or other data to be stored and retrieved. Memory device 202 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 202 may be configured to store, without limitation, application source code, application object code, source code portions of interest, object code portions of interest, configuration data, execution events and/or any other type of data.

Computing device 200, in the illustrated embodiment, includes a communication interface 206 coupled to processor 204. Communication interface 206 communicates with one or more remote devices, such as a clinician or patient programmer. To communicate with remote devices, communication interface 206 may include, for example, a radio-frequency (RF) adapter, such as a Bluetooth or medical implant communication system (MICS) adapter, and/or a near-field telecommunications adapter, also referred to as inductive telemetry.

Those of skill in the art will appreciate that power in a digital circuit is directly proportional to the amount of toggling, or frequency, at which the circuit operates. If toggling is stopped, with the exception of relatively small amounts of leakage power that are consumed just by being powered, the circuit generally does not consume power.

Further, conventional analog-to-digital converters (ADC), also referred to Nyquist converters, use fixed time steps to quantize an input analog signal. Another type of ADC, referred to as a level sampling converter, or asynchronous ADC, samples data based on crossing amplitudes. The systems and methods described herein utilize a hybrid of the two types of ADCs to save power and memory at a system and architecture level, as described herein.

Notably, for most biological data signals, the signal only has clinical interest when it changes relatively significantly from one time to another. Data that varies within a relatively small amplitude window generally has little clinical relevance. Accordingly, in the systems and methods described herein, if data falls within an amplitude window, further downstream processing and storing of that data is inhibited, thereby conserving power.

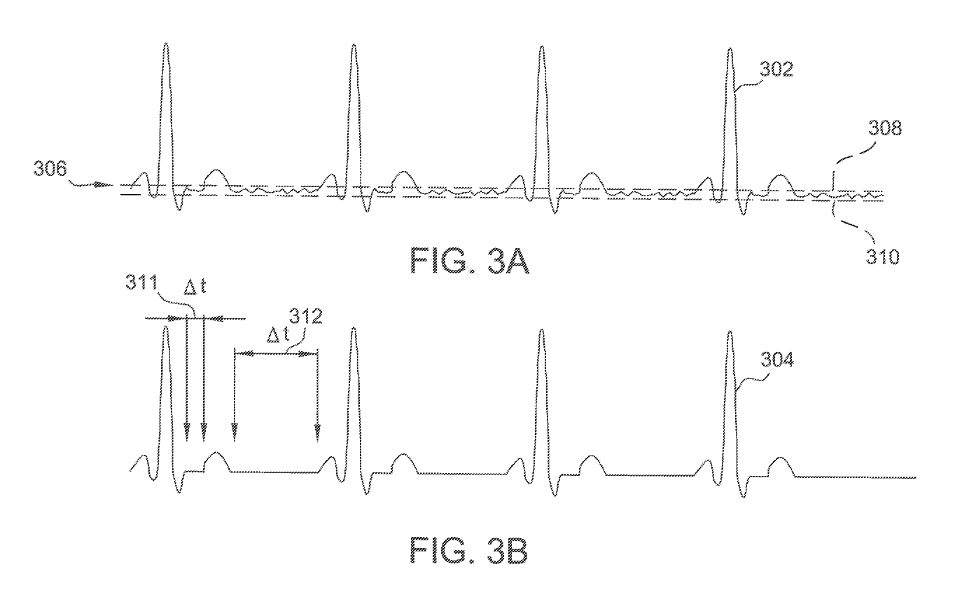

In the embodiments described herein, filtering component 104 filters raw biological data signals by ignoring (La, not processing and/or storing) signal values that fall within a defined amplitude window. For example, FIG. 3A shows an example raw biological data signal 302, and FIG. 3B shows a filtered biological data signal 304 that is generated by filtering raw biological data signal 302 using filtering component 104. Raw biological data signal 302 is detected, or acquired, by sensing component 102. In the example of FIGS. 3A and 3B, the biological data signal is an electrocardiogram (ECG). Alternatively, the biological data signal may be any signal capable of being processed using the systems and methods described herein.

In FIG. 3A, an example amplitude window 306 is overlaid on raw biological data signal 302. Amplitude window 306 is defined by an upper threshold 308 and a lower threshold 310. As shown in FIG. 3B, values of raw biological data signal 302 that fall within amplitude window 306 values greater than or equal to lower threshold 310 and less than or equal to upper threshold 308) are effectively ignored in filtered biological data signal 304. Specifically, values that fall within amplitude window 306 are not subjected to the additional processing and/or storage that values falling outside of amplitude window 306 are subjected to.

In FIG. 3A, amplitude window 306 has a fixed size and position (i.e., upper and lower thresholds 308 and 310 remain constant over time and amplitude window 306 is centered on a baseline of raw biological data signal 302). However, in some embodiments, the size and/or position of amplitude window 306 varies dynamically over time. For example, for some biological data signals, the baseline value changes over time. As such, for such signals, the size and/or position of amplitude window 306 may vary over time as appropriate.

Although the values falling within amplitude window 306 are ignored, the relative timing of events in the biological data signals still needs to be preserved. Accordingly, in one embodiment, a timer or counter (e.g., implemented using processor 204) tracks time intervals during which the biological data signal falls within amplitude window 306. The lengths of these time intervals are processed and/or stored as appropriate to facilitate analyzing the biological data signal. For example, FIG. 36 shows a first time interval 311 and a second time interval 312 that would be tracked and stored.

In one embodiment, filtering component 104 ignores signal values that fall within amplitude window 306 by not converting (using an ADC) signal values that fall within amplitude window 306. Because the ADC is not converting, the digital output of the ADC is not toggling and not consuming power. However, once the signal crosses upper threshold 308 or lower threshold 310, the ADC is enabled and starts converting the signal.

While the signal remains within amplitude window 306, the output (i.e., filtered biological data signal 304) is set to a fixed value in one embodiment. The fixed value may be, for example, a pre-programmed value, or the last acquired value before the signal entered amplitude window 306. As long the signal remains within amplitude window 306, no new conversion values are generated. As described above, once the signal exits amplitude window 306, the ADC is enabled and resumes converting.

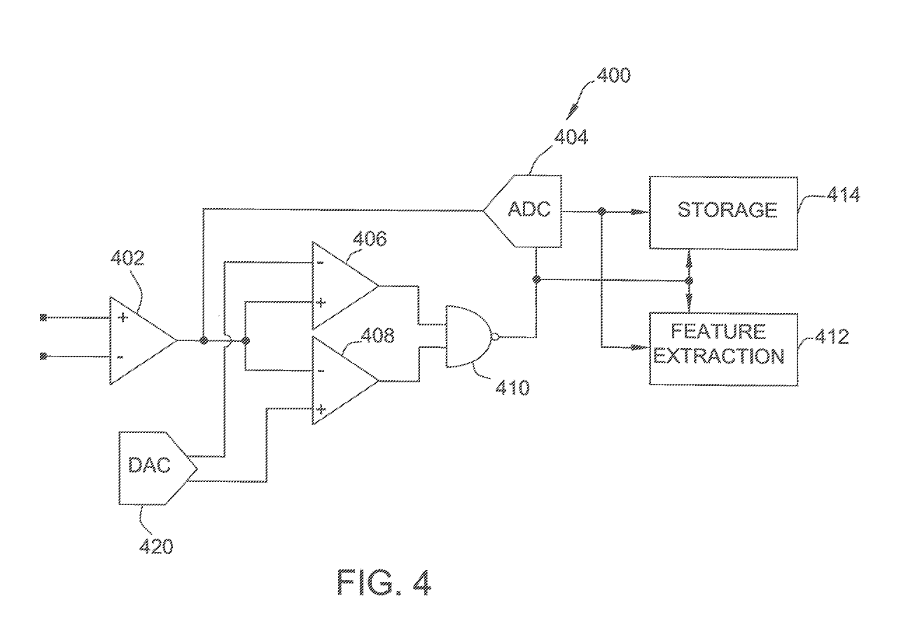

FIG. 4 is a circuit diagram of one embodiment of a filtering architecture 400 that may be used to implement filtering component 104 of IND 100 (both shown in FIG. 1). Filtering architecture 400 includes a sensing amplifier 402 that amplifies the raw biological data signal received from sensing component 102. The amplified signal is provided to an analog-to-digital converter (ADC) 404 and first and second comparators 406 and 408. First and second comparators 406 and 408 compare the amplified signal to the upper and lower thresholds defining the amplitude window, and output corresponding signals to an AND gate 410.

If the amplified signal is both i) greater than or equal to the lower threshold and ii) less than or equal to the upper threshold, AND gate 410 outputs a signal to ADC 404 that inhibits operation of ADC 404. If, however, the amplified signal falls outside of the amplitude window, AND gate 410 does not inhibit operation of ADC 404, and feature extraction 412 (e.g., processing the signal to identify artifacts, further filtering the signal, etc.) and storage 414 operations are performed on the converted signal. Accordingly, the comparison of the signal to the upper and lower thresholds is performed upstream of ADC 404.

In this embodiment, the upper and lower thresholds are controlled using a digital-to-analog converter 420. Accordingly, the size and position of the amplitude window may be user-defined (e.g., by user input on a programming device in communication with IMD 100).

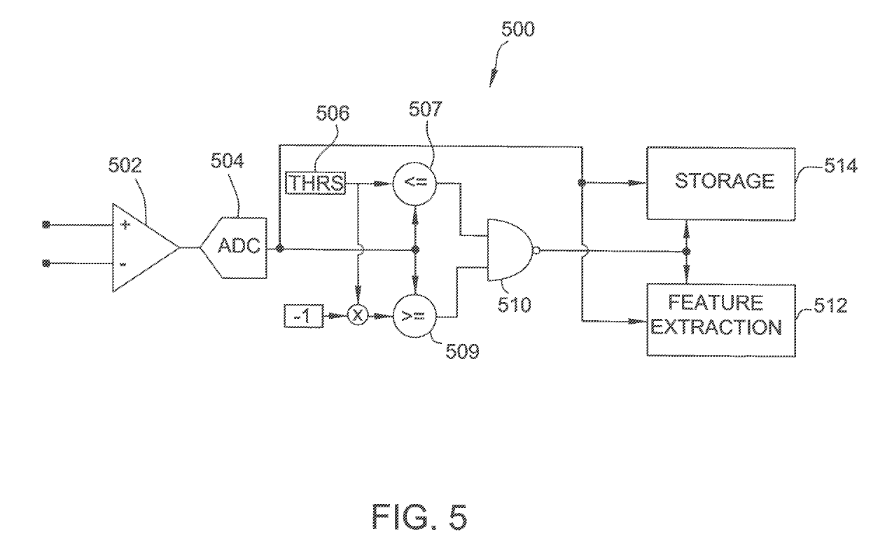

FIG. 5 is a circuit diagram of another embodiment of a filtering architecture 500 that may be used to implement filtering component 104 of IMD 100 (both shown in FIG. 1). Filtering architecture 500 includes a sensing amplifier 502 that amplifies the raw biological data signal received from sensing component 102. In this embodiment, in contrast to filtering architecture 400, an analog-to-digital converter (ADC) 504 converts all signal values, and the determination as to whether the signal value falls within the amplitude window is made after the conversion. Accordingly, the comparison of the signal to the amplitude window is made in the digital domain, instead of in the analog domain. Further, the comparison of the signal to the upper and lower thresholds is performed downstream from ADC 504.

Specifically, in this embodiment, filtering architecture 500 determines whether the output of ADC 504 is ii) less than or equal to a threshold value 506 (corresponding to the upper threshold) using a first digital comparator 507 and ii) greater than or equal to an inverse of threshold value 506 (corresponding to the lower threshold) using a second digital comparator 509. If both of these conditions are satisfied, an AND gate 510, in response to receiving corresponding signals, outputs a signal that inhibits feature extraction 512 and storage 514 operations from being carried out on the signal output by sensing amplifier 502. In the embodiment of FIG. 5, the amplitude window is centered around a zero value. However, those of skill in the art will appreciate that amplitude window may have any suitable width or position.

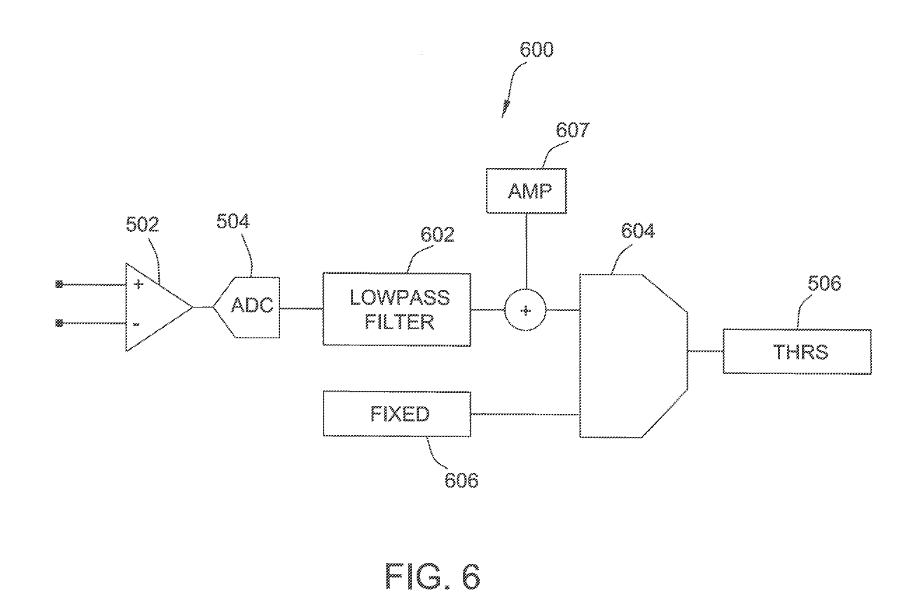

The threshold values used in filtering architectures 400 and 500 may be a fixed value or dynamic value. That is, in some embodiments, the threshold values may dynamically track a DC offset value of the raw biological data signal. For example, FIG. 6 is a circuit diagram for a threshold determination circuit 600 that may be used to determine threshold value 506.

In the embodiment shown in FIG. 6, the output of ADC 504 is filtered by a low pass filter 602 to generate a signal that corresponds to an average baseline value of the biological signal. The baseline value may change over time due to the type of signal and/or external conditions. Accordingly, threshold value 506 is adaptable to changes in the baseline signal.

A selection element 604 selects between the signal output by low pass filter 602 and a fixed value 606, and the value selected by selection element 604 is used to calculate threshold value 506. The operation of selection element 604 may be pre-programmed or user configurable. As shown in FIG. 6, an offset amplitude 607 is applied to the signal output by low pass filter 602 prior to that signal being received at selection element 604. This results in the amplitude window being set a fixed distance from the baseline value.

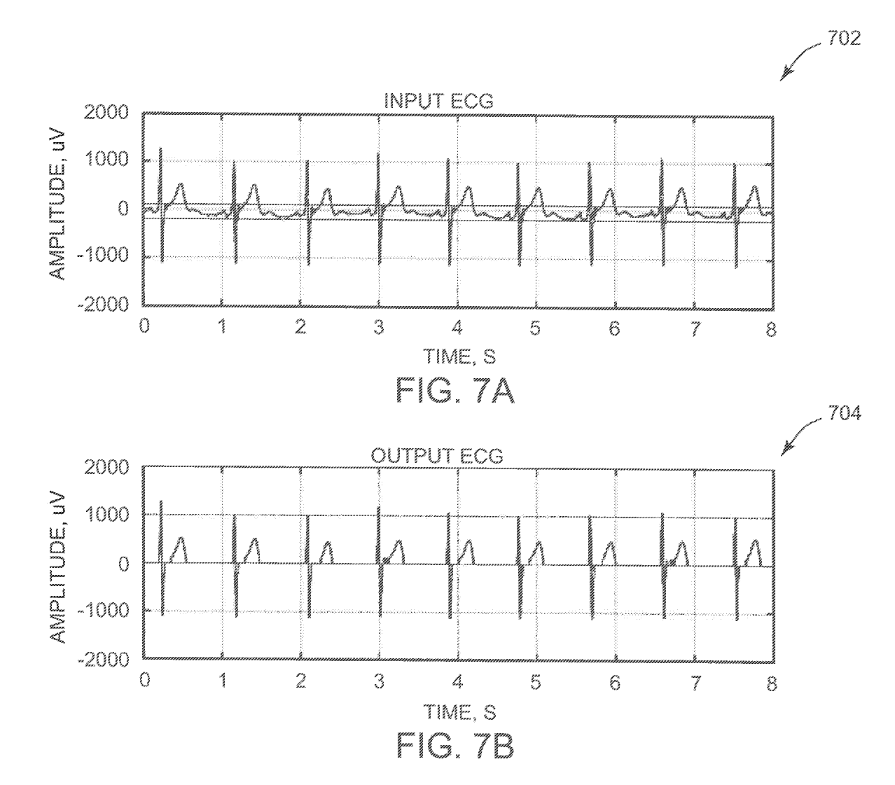

Depending on the size of the amplitude window, using the embodiments described herein, a relatively large amount of power savings may be realized. For example, FIGS. 7A and 7B are plots of an example raw ECG signal 702 and a filtered ECG signal 704, respectively. In this example, raw ECG signal 702 falls within an amplitude window 706 approximately 72% of the time. Accordingly, using the systems and methods described herein would equate to an approximately 72% reduction in signal toggling.

Accordingly, the systems and method described herein facilitate efficiently processing biological data signals. An implantable medical device includes a sensing component that acquires a raw biological data signal. A filtering component communicatively coupled to the sensing component filters the raw biological data signal to generate a filtered biological data signal by ignoring signal values that fall within an amplitude window defined between an upper threshold and a lower threshold. A processing and storage component communicatively coupled to the filtering component stores the filtered biological data signal.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense

What is Claimed is:

1. An implantable medical device comprising:
a sensing component configured to acquire a raw biological data signal;
a filtering component communicatively coupled to the sensing component, the filtering component configured to filter the raw biological data signal to generate a filtered biological data signal, wherein to filter the raw biological data signal, the filtering component is configured to ignore signal values that fall within an amplitude window defined between an upper threshold and a lower threshold; and
a processing and storage component communicatively coupled to the filtering component, the processing and storage component configured to store the filtered biological data signal.

2. The implantable medical device of claim 1, wherein the filtering component comprises an analog-to-digital converter (ADC).

3. The implantable medical device of claim 2, wherein the filtering component comprises:
a sensing amplifier configured to amplify the raw biological data signal;
a first comparator configured to compare a venue of the amplified signal to the upper threshold;
a second comparator configured to compare the value of the amplified signal to the lower threshold; and
an AND gate coupled to the first and second comparators, the AND gate configured to inhibit operation of the ADC when the value of the amplified signal is i) less than or equal to the upper threshold and ii) greater than or equal to the lower threshold.

4. The implantable medical device of claim 2, wherein the filtering component comprises:
a sensing amplifier configured to amplify the raw biological data signal;
the ADC configured to convert the amplified signal;
a first digital comparator configured to compare a value of the converted signal to the upper threshold;
a second digital comparator configured to compare the value of the converted signal to the lower threshold; and
an AND gate coupled to the first and second digital comparators, the AND gate configured to inhibit at least a storage operation when the value of the converted signal is i) less than or equal to the upper threshold and ii) greater than or equal to the lower threshold.

5. The implantable medical device of claim 4, wherein the upper threshold corresponds to a threshold value, and wherein the lower threshold corresponds to an inverse of the threshold value.

6. The implantable medical device of claim 5, wherein the filtering component further comprises a threshold determination circuit configured to dynamically determine the threshold value based on a DC offset of the raw biological data signal.

7. The implantable medical device of claim 1, wherein the upper threshold and the lower threshold are determined based on a user input received at a programmer device communicatively coupled to the implantable medical device.

8. The implantable medical device of claim 1, wherein the implantable medical device comprises an implantable cardiac monitor.

9. The implantable medical device of claim 8, wherein the raw biological data signal is an intra-cardiac electrocardiogram or electrocardiogram.

10. The implantable medical device of claim 1, wherein to store the filtered biological data signal, the processing and storage component is configured to:
identify at least one time interval during which signal values fall within the amplitude window; and
store data indicative of the at least one time interval.

11. A method for processing biological data signals acquired using an implantable medical device, the method comprising:
acquiring, using a sensing component of the implantable medical device, a raw biological data signal;
filtering, using a filtering component of the implantable medical device, the raw biological data signal to generate a filtered biological data signal, wherein the filtering comprises ignoring signal values that fail within an amplitude window defined between an upper threshold and a lower threshold; and
storing the filtered biological data signal using a processing and storage component of the implantable medical device.

12. The method of claim 11, wherein filtering the raw biological data signal comprises filtering the raw biological data signal using a filtering component that includes an analog-to-digital converter (ADC).

13. The method of claim 12, wherein filtering the raw biological data signal comprises:
amplifying the raw biological data signal using a sensing amplifier;
comparing a value of the amplified signal to the upper threshold using a first comparator;
comparing the value of the amplified signal to the lower threshold using a second comparator; and
inhibiting operation of the ADC when the value of the amplified signal is i) less than or equal to the upper threshold and ii) greater than or equal to the lower threshold.

14. The method of claim 12, wherein filtering the raw biological data signal comprises:
amplifying the raw biological data signal using a sensing amplifier;
converting the amplified signal using the ADC;
comparing a value of the converted signal to the upper threshold using a first digital comparator;
comparing the value of the converted signal to the lower threshold using a second digital comparator; and
inhibiting at least a storage operation when the value of the converted signal is i) less than or equal to the upper threshold and ii) greater than or equal to the lower threshold.

15. The method of claim 14, wherein the upper threshold corresponds to a threshold value, and wherein the lower threshold corresponds to an inverse of the threshold value.

16. The method of claim 15, further comprising dynamically determining the threshold value based on a DC offset of the raw biological data signal.

17. The method of claim 11, further comprising determining the upper threshold and the lower threshold are determined based on a user input received at a programmer device communicatively coupled to the implantable medical device.

18. The method of claim 11, wherein acquiring a raw biological data signal comprises acquiring a raw biological data signal using a sensing component of an implantable cardiac monitor.

19. The method of claim 18, wherein acquiring a raw biological data signal comprises acquiring an intra cardiac electrocardiogram or dectrocardiogram.

20. The method of claim 11, wherein storing the filtered biological data signal comprises:
identifying at least one time interval during which signal values fall within the amplitude window; and
storing data indicative of the at least one time interval.
